(19) United States
(12) Patent Application Publication
(10) Pub. No.: US 2008/0108547 A1
(43) Pub. Date: May 8, 2008

(54) Protease Inhibitor

(76) Inventors: Larry Edmund Taylor II, Lakewood, CO (US); Ronald M. Weiner, Potomac, MD (US); Steven W. Hutcheson, Columbia, MD (US)

Correspondence Address: Carter Deluca Farrell & Schmidt, 445 Broad Hollow Road, Suite 225, Melville, NY, 11747, US

(21) Appl. No.: 11/632,756

(22) PCT Filed: Jul. 20, 2005
(86) PCT No.: PCT/US05/25786

§ 371 Date: Oct. 1, 2007

Related U.S. Application Data
(60) Provisional application No. 60/589,516, filed on Jul. 20, 2004.

(51) Int. Cl. A61K 38/00; C07H 21/04; C12N 15/00; C12P 21/04; C07K 16/00; A61P 43/00; C12N 5/00; C12N 5/02; C12N 5/04

(52) U.S. Cl. 514/2; 536/22.1; 435/320.1; 435/325; 435/348; 435/358; 435/365; 435/366; 435/252.33; 435/255.1; 435/69.1; 530/300; 435/219; 530/387.9; 530/388.1; 530/387.3

(57) ABSTRACT

The present disclosure provides isolated nucleic acids and their resulting polypeptides from Saccharophagus degradans strain 2-40, which may be utilized as protease inhibitors.

### CROSS REFERENCE TO RELATED APPLICATIONS

This application claims the benefit and priority to U.S. Provisional Patent Application Ser. No. 60/589,516 filed on Jul. 20, 2004, the entire disclosure of which is hereby incorporated by reference herein.

### GOVERNMENT RIGHTS

The present disclosure was made with Government support under U.S. National Oceanic and Atmospheric Administration/Seagrant SA7528051E and National Science Foundation Grant DEB0109869. The Government has certain rights in the present disclosure.

### BACKGROUND

The present disclosure relates to genes and proteins produced therefrom that are capable of inhibiting proteases and, more particularly, to proteins produced from a gene found in a strain of Saccharophagus degradans.

The marine bacterium Saccharophagus degradans strain 2-40, formerly known as Microbulbifer degradans strain 2-40, was originally isolated from the salt marsh cord grass, Spartina alterniflora, in the Chesapeake Bay watershed. It is a pleomorphic, gram negative bacterium that is aerobic, rod shaped, and motile. S. degradans produces numerous proteases, lipases, and carbohydrases that allow it to degrade a variety of complex, insoluble polysaccharides of plant, fungi, and animal origin. These polysaccharides include alginate, araban, carrageenan, cellulose, chitin, glycogen, β-glucan, pectin, laminarin, pullulan, starch, xylan, and agar.

α-Macroglobulins are large (˜180 kDa) glycoproteins which function as protease-binding proteins. They are present in the bloodstream of vertebrates and invertebrates, as well as bird and reptile egg whites. Those which have been previously studied are eukaryotic in origin, and the few prokaryotic examples in genetic databases have not been characterized. α-Macroglobulin protein inhibitors are commercially available and include, for example, a tetrameric protein isolated from human plasma sold by Sigma Aldrich (St. Louis, Mo.).

One α-macroglobulin, alpha-2-macroglobulin (A2M), is a highly conserved protease inhibitor present in plasma at relatively high concentrations (2-4 mg/ml). Human A2M, which is the best-studied A2M, is a tetramer of four identical 180 kDa subunits, having a total molecular weight of about 720 kDa, that forms a hollow cylinder-like structure. All known A2Ms contain an exposed “bait region”, which is a peptide stretch with specific cleavage sites for all four classes of proteases: serine, cysteine, aspartic and metallo proteases. While other protease inhibitors may only act on one class of proteases, the A2Ms are capable of acting on all four classes of proteases. Cleavage of the bait region triggers a conformational change in the A2M, trapping the protease. Following the conformational change, a thiol-ester bond is cleaved, resulting in the covalent attachment of the protease to the A2M molecule. The “activated” A2M is now recognizable by its receptor. The receptor bound activated A2M is then endocytosed, thus removing the potentially harmful proteases from the circulation. Due to this mechanism of activity, A2Ms are not protease inhibitors in the classic sense but, instead, function as what some refer to as sophisticated “molecular traps.”

Synthetic protease inhibitors are expensive and are generally specific for a particular class of protease. Thus, mixtures of these inhibitors, sometimes referred to as cocktails, are prescribed for the treatment of various disease states, including HIV, depending upon the various proteases they are expected to encounter. A single product effective against all four protease classes could thus provide a cost-effective alternative to the expensive, and often toxic, synthetic cocktails.

### SUMMARY

The present disclosure features a nucleic acid molecule encoding a protein or polypeptide of Saccharophagus degradans which functions as a protease inhibitor. In one embodiment, the present disclosure provides an isolated nucleic acid molecule having the nucleotide sequence shown in SEQ ID NO: 1. In some embodiments, the isolated nucleic acid molecule has at least about 50% nucleic acid sequence identity to the nucleotide sequence shown in SEQ ID NO: 1.

In other embodiments, the present disclosure provides isolated nucleic acid molecules having the nucleotide sequence shown in SEQ ID NO: 5 or SEQ ID NO: 7. In some embodiments, the isolated nucleic acid molecule has at least about 50% nucleic acid sequence identity to the nucleotide sequence shown in SEQ ID NO: 5 or SEQ ID NO: 7.

Expression vectors possessing such nucleic acid molecules, and host cells having such expression vectors are also provided.

The present disclosure also provides processes for producing a polypeptide by culturing these host cells under conditions suitable for expression of said polypeptide and recovering said polypeptide from the cell culture.

The present disclosure also provides a peptide having the amino acid sequence of SEQ ID NO: 2. In some embodiments, the peptide has at least about 30% amino acid sequence identity to the amino acid sequence of SEQ ID NO: 2 and possess alpha-2-macroglobulin like activity. In other embodiments, the peptide comprises a conservative substitution variant of the amino acid sequence having at least about 30% amino acid sequence identity to the amino acid sequence of SEQ ID NO: 2.

In other embodiments, present disclosure also provides a peptide having the amino acid sequence of SEQ ID NO: 6 or SEQ ID NO: 8. In some embodiments, the peptide has at least about 30% amino acid sequence identity to the amino acid sequence of SEQ ID NO: 6 or SEQ ID NO: 8 and possess alpha-2-macroglobulin like activity. In other embodiments, the peptide comprises a conservative substitution variant of the amino acid sequence having at least about 30% amino acid sequence identity to the amino acid sequence of SEQ ID NO: 6 or SEQ ID NO: 8.

Antibodies to such peptides are also disclosed.

Pharmaceutical compositions including such peptides and methods for treating diseases with such peptides are also disclosed.

Methods for utilizing such peptides to remove proteases from cultures, including fermentation cultures and cultures utilized in the expression and purification of recombinant proteins, are also provided.

### BRIEF DESCRIPTION OF THE DRAWINGS

Various embodiments of the present disclosure will be described herein below with reference to the figures wherein:

FIGS. 1A-C are the nucleotide sequence of the AmgA gene, i.e., SEQ ID NO: 1;

FIG. 2 is the amino acid sequence of the AmgA polypeptide, i.e., SEQ ID NO: 2;

FIGS. 3A-D are the nucleotide sequence of a HisTag® expression vector having the AmgA gene incorporated therein (referred to as pLTAmgA001), i.e., SEQ ID NO: 3;

FIG. 4 is the amino acid translation sequence of the open reading frame (ORF) for the peptide obtained from the HisTag® expression vector having the AmgA gene incorporated therein, which is referred to as AmgA:His₆ (bases 278 to 5285 of pLTAmgA001), i.e. SEQ ID NO: 4;

FIG. 5 is the nucleotide sequence of a portion of the AmgA gene corresponding to the core region, containing protease bait sequences (bases 844-2664), i.e., SEQ ID NO: 5;

FIG. 6 is the amino acid sequence of a portion of the AmgA peptide corresponding to the core region, which contain protease bait sequences, (amino acids 282-888), i.e., SEQ ID NO: 6;

FIG. 7 is the nucleotide sequence of a portion of the AmgA gene corresponding to the complement protein C4-like region (bases 3451 to 4227; codons for thiol ester cysteine and glutamine are at bases 3490-3493 and 3499-3501), i.e., SEQ ID NO: 7; and

FIG. 8 is the amino acid sequence of a portion of the AmgA polypeptide corresponding to the complement protein C4-like region, (amino acids 1151 to 1409), i.e., SEQ ID NO: 8.

### DETAILED DESCRIPTION OF EMBODIMENTS

U.S. Pat. Nos. 5,418,156 and 6,759,040, the entire disclosures of each of which are incorporated by reference herein, disclose enzyme systems and enzyme mixtures, respectively, from Microbulbifer degradans strain 2-40. U.S. Patent Application Nos. 20050112750 and 20050003503, the entire disclosures of each of which are incorporated by reference herein, disclose polynucleotides and degradative enzymes from Microbulbifer degradans strain 2-40.

In accordance with the present disclosure, a gene encoding a 1,637 amino acid protein with significant sequence similarity to α-macroglobulins has been found in the genome of Saccharophagus degradans strain 2-40, formerly known as Microbulbifer degradans strain 2-40. This gene, and its resulting peptide, are referred to herein as α-macroglobulin A, or AmgA.

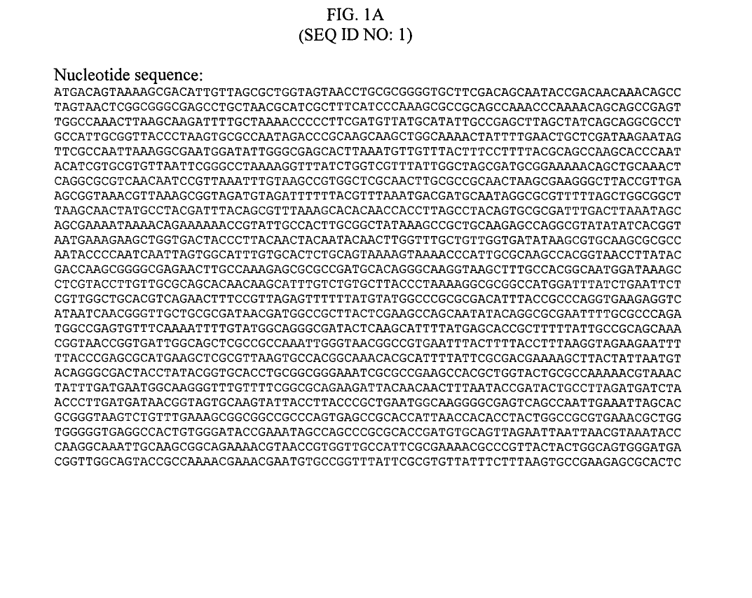

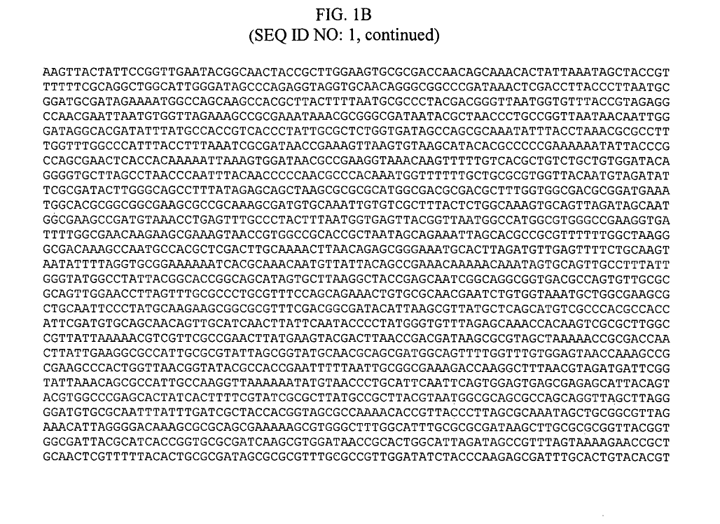

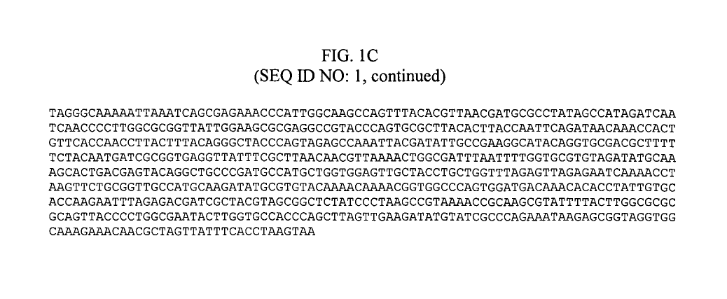

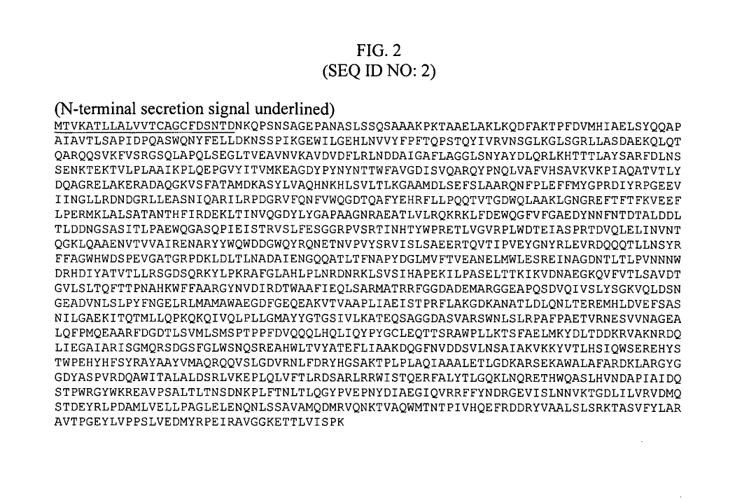

The present disclosure includes both an isolated or purified nucleic acid molecule encoding AmgA, having a nucleotide sequence of SEQ ID NO: 1 (FIGS. 1A-1C), and the AmgA polypeptide encoded thereby, having a sequence of SEQ ID NO: 2 (FIG. 2). Also encompassed by the present disclosure are fragments of such nucleic acid molecules and fragments of such AmgA polypeptide, e.g., a biologically active portion of an AmgA protein. The AmgA nucleic acid and protein molecules of the present disclosure play a role in or function in the inhibition of proteases.

Nucleic acid molecules encoding such polypeptides or proteins are collectively referred to as “nucleic acids” or “AmgA nucleic acids.” As used herein, the term “nucleic acid molecule” includes DNA molecules (for example, a cDNA or genomic DNA) and RNA molecules (such as mRNA) and analogs of the DNA or RNA generated, for example, by the use of nucleotide analogs. The nucleic acid molecule can be single-stranded or double-stranded.

The nucleotide sequence and conceptual translation of AmgA were deposited into the RefSeq database under the accession number ZP_{—}00065728.1. This was done Jun. 28, 2002 as part of the Microbulbifer degradans stain 2-40 whole genome shotgun sequencing project, conducted by the Department of Energy Joint Genome Initiative (DOE/JGI). The organism has been redeposited into the American Type Culture Collection (ATCC, Manassas, Va.) as ATCC 43961. The genome was recently finished and the final assembly, dated Jan. 19, 2005 is available on the World Wide Web at http:/genome.ornl.gov/microbial/mdeg/. In this version of the assembly, AmgA is designated gene 523, and has the accession numbers gi 48861694 and ZP_{—}00315594.

In some useful embodiments, the nucleic acid molecule may be in isolated form and may be DNA such as cDNA or genomic DNA. The DNA may encode the same amino acid sequence as naturally occurring AmgA or an AmgA derivative. The nucleotide sequence may correspond to the genomic coding sequence (including exons and introns) or to the nucleotide sequence in cDNA from mRNA transcribed from the genomic gene, or it may carry one or more nucleotide substitutions, deletions and/or additions thereto.

An “isolated” or “purified” nucleic acid or nucleotide is substantially free of cellular material or other contaminating proteins from the cell or tissue source from which the nucleic acid is derived. For example, with regards to genomic DNA, the term “isolated” includes nucleic acid molecules which are separated from the chromosome with which the genomic DNA is naturally associated. In embodiments, an “isolated” nucleic acid is free of sequences which naturally flank the nucleic acid (i.e., sequences located at the 5′ and/or 3′ ends of the nucleic acid) in the genomic DNA of the organism from which the nucleic acid is derived, typically Saccharophagus degradans. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

The terms “derivatives” or “derivative”, whether in relation to a nucleic acid molecule or a protein, includes parts, mutants, fragments, and analogues, as well as hybrid or fusion molecules and glycosylation variants. Particularly useful derivatives may include single or multiple amino acid substitutions, deletions and/or additions to the AmgA amino acid sequence.

In one embodiment, an isolated nucleic acid molecule of the present disclosure includes the nucleotide sequence shown in SEQ ID NO: 1, or a portion of any of this nucleotide sequence. The nucleic acid molecule may include sequences encoding the AmgA protein from Saccharophagus degradans (i.e., “the coding region”, not including the terminal codon), as well as 5′ untranslated sequences. Alternatively, the nucleic acid molecule can include only the coding region of SEQ ID NO: 1 with no flanking sequences which normally accompany the subject sequence. In one embodiment, the nucleic acid molecule encodes a sequence corresponding to the mature protein of SEQ ID NO: 2.

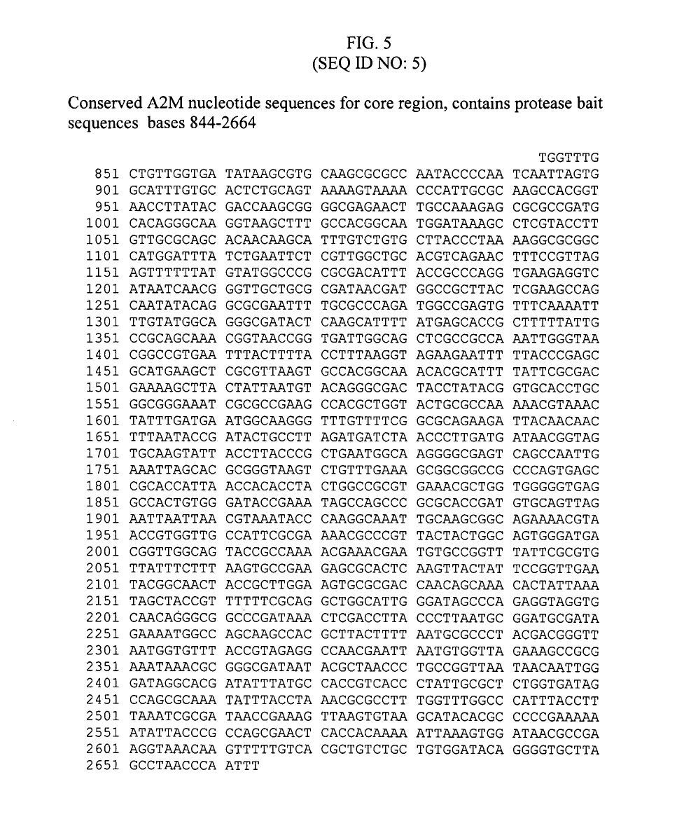

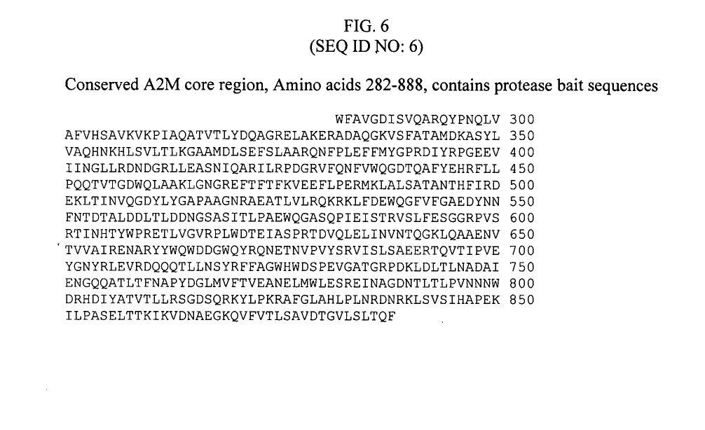

In some embodiments, an isolated nucleic acid molecule of the present disclosure includes the nucleotide sequence shown in SEQ ID NO: 5, or any portion of this nucleotide sequence. The nucleotide sequence shown in SEQ ID NO: 5 (set forth as FIG. 5) is bases 844-2664 of SEQ. ID NO: 1, which corresponds to the core region of the AmgA gene possessing protease bait sequences. In one embodiment, the nucleic acid molecule of SEQ ID NO: 5 encodes a peptide corresponding to the conserved A2M core region of the AmgA polypeptide as set forth in SEQ ID NO: 6 (which, in turn, is set forth as FIG. 6). SEQ ID NO: 6 is amino acids 282-888 of SEQ. ID NO: 2, and contains protease bait sequences of the AmgA polypeptide.

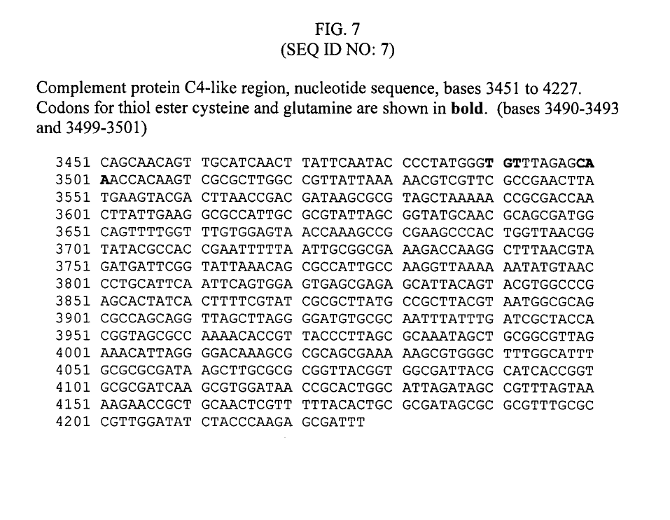

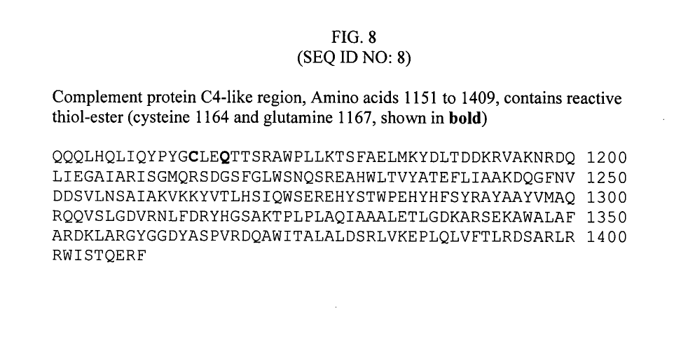

In other embodiments, an isolated nucleic acid molecule of the present disclosure includes the nucleotide sequence shown in SEQ ID NO: 7, or any portion of this nucleotide sequence. The nucleotide sequence shown in SEQ ID NO: 7 (set forth as FIG. 7) is bases 3451 to 4227 of SEQ. ID NO: 1, which corresponds to the complement protein C4-like region of the AmgA gene. Codons for thiol ester cysteine and glutamine are shown in bold in FIG. 7 (bases 3490-3493 and 3499-3501). In one embodiment, the nucleic acid molecule encodes a sequence corresponding to the complement protein C4-like region of the AmgA polypeptide as set forth in SEQ ID NO: 8 (which, in turn, is set forth as FIG. 8). SEQ ID NO: 8 is amino acids 1151 to 1409 of SEQ. ID NO: 2, and contains reactive thiol-esters (cysteine 1164 and glutamine 1167, both shown in bold in FIG. 8).

In another embodiment, an isolated nucleic acid molecule of the present disclosure includes a nucleic acid molecule which is a complement of the nucleotide sequence shown in SEQ ID NO: 1, 5 or 7, or a portion of any of these nucleotide sequences. In other embodiments, the nucleic acid molecule of the present disclosure is sufficiently complementary to the nucleotide sequence shown in SEQ ID NO: 1, 5 or 7 such that it can hybridize to the nucleotide sequence shown in SEQ ID NO: 1, 5 or 7, thereby forming a stable duplex.

In some embodiments, an isolated nucleic acid molecule of the present disclosure that hybridizes under stringent conditions to the sequence of SEQ ID NO: 1, 5 or 7 corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein). In typically useful embodiments, nucleic acids include a nucleotide sequence capable of hybridizing under stringent hybridization conditions to a nucleic acid molecule of SEQ ID NO: 1, 5 or 7.

As used herein, the term “hybridizes under stringent conditions” describes conditions for hybridization and washing. Stringent conditions are within the purview of those skilled in the art and can be found in Current Protocols in Molecular Biology, John Wiley & Sons, New York (1989), 6.3.1-6.3.6. Aqueous and nonaqueous methods are described in that reference and either can be used. An example of stringent hybridization conditions are hybridization in about 6× sodium chloride/sodium citrate (SSC) at about 45° C., followed by one or more washes in about 0.2×SSC, about 0.1% SDS at a temperature from about 50° C. to about 70° C. In some embodiments, stringent hybridization conditions may be hybridization in about 6× sodium chloride/sodium citrate (SSC) at about 45° C., followed by one or more washes in about 0.2×SSC, about 0.1% SDS at about 65° C. In other embodiments, stringency conditions include about 0.5M Sodium Phosphate, about 7% SDS at about 65° C., followed by one or more washes at about 0.2×SSC, about 1% SDS at about 65° C.

Isolated nucleic acids of the present disclosure have a nucleotide sequence sufficiently homologous to SEQ ID NO: 1 or a fragment thereof, for example SEQ ID NOs: 5 or 7. Similarly, isolated proteins of the present disclosure have an amino acid sequence sufficiently homologous to the amino acid sequence of SEQ ID NO: 2 or a fragment thereof, for example SEQ ID NOs: 6 or 8. As used herein, the term “sufficiently homologous” refers to a first amino acid or nucleotide sequence which contains a sufficient or minimum number of identical or equivalent (e.g., an amino acid residue which has a similar side chain) amino acid residues or nucleotides to a second amino acid or nucleotide sequence, respectively, such that the first and second amino acid or first and second nucleotide sequences share common structural domains or motifs and/or a common functional activity. Those sequences having an equivalent amino acid residue or nucleotide sequence are sometimes referred to herein as a conservative substitution variant.

Where the AmgA nucleotide sequence or the amino acid sequence of the AmgA peptide includes a conservative substitution variant, the resulting AmgA peptide will possess a conservative amino acid substitution. Such conservative substitution variants are still deemed sufficiently homologous to the sequences of the present disclosure. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in an AmgA protein is typically replaced with another amino acid residue from the same side chain family. Alternatively, in another embodiment, mutations can be introduced randomly along all or part of an AmgA coding sequence, such as by saturation mutagenesis, and the resultant mutants can be screened for AmgA biological activity to identify mutants that retain activity. Following mutagenesis of SEQ ID NO: 1, 5 or 7, the encoded protein can be expressed recombinantly and the activity of the protein can be determined.

For example, amino acid or nucleotide sequences which share common structural domains and have at least about 30% to about 100% homology, in some embodiments about 60% to about 95% homology, in other embodiments about 70% to about 90% homology across the amino acid or nucleotide sequences of the domains, respectively, are defined herein as sufficiently homologous. Furthermore, amino acid or nucleotide sequences which share a common functional activity and have at least about 30% to about 100% homology, in some embodiments about 60% to about 95% homology, in other embodiments about 70% to about 90% homology across the amino acid or nucleotide sequences of the domains, respectively, are defined herein as sufficiently homologous.

In some embodiments, a nucleic acid is sufficiently homologous if it has at least about 50% nucleic acid sequence identity to the nucleotide sequences of SEQ ID NO: 1, 5 or 7, or is a conservative substitution variant thereof; in some embodiments it has at least about 65% nucleic acid identity to the nucleotide sequences of SEQ ID NO: 1, 5 or 7, or is a conservative substitution variant thereof; in other embodiments it has at least about 80% nucleic acid sequence identity to the nucleotide sequence of SEQ ID NO: 1, 5 or 7, or is a conservative substitution variant thereof. Similarly, in some embodiments, a peptide is sufficiently homologous if it has at least about 30% amino acid sequence identity to the amino acid sequences of SEQ ID NO: 2, 6 or 8, or is a conservative substitution variant thereof; in some embodiments it has at least about 55% amino acid sequence identity to the amino acid sequences of SEQ ID NO: 2, 6 or 8, or is a conservative substitution variant thereof; in other embodiments it has at least about 80% amino acid sequence identity to the amino acid sequence of SEQ ID NO: 2, 6 or 8, or is a conservative substitution variant thereof.

The homologous nucleotide sequences or amino acid sequences possess a common functional activity, in some embodiments alpha-2-macroglobulin like activity. As used herein, a peptide possesses alpha-2-macroglobulin like activity where it is capable of binding at least one class of protease, i.e. serine, cysteine, aspartic and/or metallo protease.

Amino acid and nucleotide sequences can be evaluated for homology either manually by one skilled in the art, or by using computer-based sequence comparison and identification tools that employ algorithms such as BLAST (Basic Local Alignment Search Tool; Altschul et al. (1993) J. Mol. Biol. 215:403410). In general, a sequence of ten or more contiguous amino acids or thirty or more contiguous nucleotides may be necessary in order to putatively identify a polypeptide or nucleic acid sequence as homologous to a known protein or gene. Moreover, with respect to nucleotide sequences, gene-specific oligonucleotide probes comprising 30 or more contiguous nucleotides may be used in sequence-dependent methods of gene identification (e.g., Southern hybridization) and isolation (e.g., in situ hybridization of bacterial colonies or bacteriophage plaques). In addition, short oligonucleotides of 12 or more nucleotides may be used as amplification primers in PCR in order to obtain a particular nucleic acid fragment comprising the primers. A nucleotide sequence herein thus includes a nucleotide sequence that will afford specific identification and/or isolation of a nucleic acid fragment which includes the sequence. The present disclosure provides for amino acid and nucleotide sequences encoding polypeptides that encompass one or more particular prokaryotic proteins. The skilled artisan, having the benefit of the sequences as reported herein, may now use all or a portion of the disclosed sequences for purposes within the purview of those skilled in this art, including identifying homologous genes and peptides. Accordingly, the present disclosure includes the complete sequences as reported herein, as well as homologous sequences as described above.

In another embodiment, a nucleic acid molecule includes a nucleotide sequence that includes part, or all, of the coding region and extends into either (or both) the 5′ or 3′ noncoding region. Other embodiments include a fragment which includes a nucleotide sequence encoding an amino acid fragment described herein. Nucleic acid fragments can encode a specific domain or site described herein or fragments thereof, particularly fragments thereof which are at least about 150 amino acids in length. Fragments also include nucleic acid sequences corresponding to specific amino acid sequences described above or fragments thereof.

A nucleic acid fragment can include a sequence corresponding to one or more domains, regions, or functional sites described herein and can encode an epitope bearing region of a polypeptide described herein. Thus, for example, the nucleic acid fragment can include a protease binding domain, e.g., serine protease, cysteine protease, aspartic protease, metallo protease, or any combination of more than one such protease.

A nucleic acid fragment encoding a “biologically active portion of an AmgA polypeptide” can be prepared by isolating a portion of the nucleotide sequence of SEQ ID NO: 1, which encodes a polypeptide having an AmgA activity (e.g., the biological activities of the AmgA proteins as described herein), expressing the encoded portion of the AmgA protein (e.g., by recombinant expression in vitro) and assessing the activity of the encoded portion of the AmgA protein. For example, a nucleic acid fragment encoding a biologically active portion of AmgA typically includes a binding domain for at least one protease. Examples of such fragments include the nucleotide sequences set forth in SEQ ID NO: 5, which corresponds to the portion of the AmgA gene encoding the core region of the AmgA polypeptide possessing protease bait sequences, and SEQ ID NO: 7, which corresponds to the portion of the AmgA gene encoding the complement protein C4-like region of the AmgA polypeptide.

Nucleic acid variants are also included in the present disclosure. Variants can be naturally occurring, such as allelic variants (same locus), homologs (different locus), and orthologs (different organism) or can be non-naturally occurring. Non-naturally occurring variants can be made by mutagenesis techniques, including those applied to polynucleotides, cells, or organisms. The variants can contain nucleotide substitutions, deletions, inversions and insertions. Variation can occur in either or both the coding and non-coding regions. The variations can produce both conservative and non-conservative amino acid substitutions (as compared in the encoded product).

Orthologs, homologs, and allelic variants can be identified using methods known in the art. These variants comprise a nucleotide sequence encoding a polypeptide that is about 50%, in embodiments from about 55% to about 95%, typically from about 70% to about 90%, more typically from about 75% to about 85% or more identical to the amino acid sequence shown in SEQ ID NO: 2 or a fragment of this sequence, including those set forth in SEQ ID NO: 6 and SEQ ID NO: 8. Such nucleic acid molecules can readily be obtained as being able to hybridize under stringent conditions to the nucleotide sequence shown in SEQ ID NO: 1, or a fragment of this sequence, including those set forth as SEQ ID NO: 5 and SEQ ID NO: 7. Nucleic acid molecules corresponding to orthologs, homologs, and allelic variants of the nucleic acids of the present disclosure can further be isolated by mapping to the same chromosome or locus as the AmgA gene. Typical variants include those that retain binding activity to at least one type of protease.

In some embodiments, the present disclosure also provides nucleic acid fragments suitable for use as a hybridization probe. These fragments can be used, e.g., to identify a nucleic acid molecule encoding a polypeptide of the present disclosure, AmgA, and fragments suitable for use as primers, e.g., PCR primers for the amplification or mutation of nucleic acid molecules.

For example, such a nucleic acid molecule can include a fragment which can he used as a probe or primer or a fragment encoding a portion of an AmgA protein, e.g., an immunogenic or biologically active portion of an AmgA protein. A fragment can include portions of the nucleotides of SEQ ID NO: 1 which encode a fragment of AmgA. Examples of such fragments include those set forth in SEQ ID NO: 5 and SEQ ID NO: 7. The nucleotide sequence determined from the cloning of the AmgA gene allows for the generation of probes and primers designed for use in identifying and/or cloning other AmgA family members, or fragments thereof, as well as AmgA homologues, or fragments thereof, from other species.

In some embodiments, a probe/primer is an isolated or purified oligonucleotide. The oligonucleotide typically includes a region of nucleotide sequence that hybridizes under stringent conditions to at least from about 7 to about 75, in embodiments from about 15 to about 65, more typically from about 25 to about 55 consecutive nucleotides of a sense or antisense sequence of SEQ ID NO: 1, or of a naturally occurring allelic variant or mutant of SEQ ID NO: 1.

In one embodiment the nucleic acid is a probe which is from about 5 to about 200, typically from about 10 to about 100, more typically from about 15 to about 50, base pairs in length. The probe should be identical, or differ by from about 1 to about 10 bases, from a sequence disclosed herein. If alignment is needed for this comparison the sequences should be aligned for maximum homology. “Looped” out sequences from deletions or insertions, or mismatches, are considered differences.

In another embodiment a set of primers is provided, e.g., primers suitable for use in a PCR, which can be used to amplify a selected region of an AmgA sequence, e.g., a region described herein. The primers should be at least 5 to about 200, typically from about 10 to about 100, more typically from about 15 to about 50, base pairs in length. The primers should be identical, or differ by one base from a sequence disclosed herein or from a naturally occurring variant.

The present disclosure also provides detectably labeled oligonucleotide primer and probe molecules. Typically, such labels are chemiluminescent, fluorescent, radioactive, or calorimetric.

The AmgA protein of the present disclosure has an amino acid sequence shown in SEQ ID NO: 2. The molecular weight of the AmgA peptide is about 183 kDa, comparable to the known prokaryotic A2M proteins, but unlike mammalian A2M, AmgA does not form a tetramer. In view of the vast amount of cellular resources that Saccharophagus degradans strain 2-40 invests in carbohydrase synthesis, the role of protein produced by AmgA may be to protect these enzymes from proteolytic attack.

The present disclosure also includes isolated peptides having an amino acid sequence sufficiently homologous to the amino acid sequence of SEQ ID NO: 2. Such peptides may contain one or more changes in amino acid sequence, e.g., a change in an amino acid residue which is not essential for activity. Thus, such AmgA proteins differ in amino acid sequence from SEQ ID NO: 2, yet retain biological activity. Moreover, other biologically active portions, in which other regions of the protein are deleted, can be prepared by recombinant techniques and evaluated for one or more of the functional activities of a native AmgA protein. For example, one biologically active portion of the AmgA protein is set forth in SEQ ID NO: 6, which corresponds to the core region of AmgA polypeptide and possesses protease bait sequences. Another biologically active portion of the AmgA protein is set forth in SEQ ID NO: 8, which corresponds to the complement protein C4-like region of the AmgA polypeptide and contains reactive thiol-esters (cysteine and glutamine).

An “isolated” or “purified” polypeptide or protein is substantially free of cellular material or other contaminating proteins from the cell or tissue source from which the protein is derived, or substantially free from chemical precursors or other chemicals when chemically synthesized.

In one embodiment, “substantially free” means preparation of the AmgA protein having from about 30% to about 2%, in embodiments about 5%, by dry weight of non-AmgA protein (also referred to herein as a “contaminating protein”), or of chemical precursors or non-AmgA chemicals. When the AmgA protein or biologically active portion thereof is recombinantly produced, it is also typically substantially free of culture medium, that is, culture medium represents from about 20% to about 0.5%, in embodiments about 5%, of the volume of the protein preparation. The present disclosure also includes isolated or purified preparations of AmgA protein of at least about 0.01 to about 10 milligrams in dry weight.

Accordingly, another embodiment of the present disclosure features isolated AmgA proteins and polypeptides having AmgA activity. As used herein, “AmgA activity”, “biological activity of AmgA” or “functional activity of AmgA”, refer to an activity exerted by an AmgA protein, polypeptide or nucleic acid molecule on, for example, an AmgA-responsive cell or on an AmgA substrate, e.g., a nucleoside substrate, as determined in vivo or in vitro. In one embodiment, an AmgA activity is a direct activity, such as association with a protease target molecule. A “target molecule” or “binding partner” is a molecule with which an AmgA protein binds or interacts in nature, e.g., a protease such as a serine protease, cysteine protease, aspartic protease, metallo protease and/or a combination of more than one protease from these classes of proteases. An AmgA activity can also be an indirect activity, for example, a cellular signaling activity mediated by interaction of the AmgA protein with a protease.

As used herein, a “biologically active portion” of an AmgA protein also includes a fragment of an AmgA protein which participates in an interaction between an AmgA molecule and a non-AmgA molecule. Biologically active portions of an AmgA protein include peptides comprising amino acid sequences sufficiently homologous to or derived from the amino acid sequence of the AmgA protein, e.g., the amino acid sequence shown in SEQ ID NO: 2, which include less amino acids than the full length AmgA proteins, and exhibit at least one activity of an AmgA protein. Examples of such biologically portions include, but are not limited to, the amino acid sequences set forth in SEQ ID NO: 6 and SEQ ID NO: 8. Typically, biologically active portions comprise a domain or motif with at least one activity of the AmgA protein, e.g., ability to bind one of the four classes of proteases. A biologically active portion of an AmgA protein can be a polypeptide which is, for example, 10, 25, 50, 100, 200 or more amino acids in length. Biologically active portions of an AmgA protein can be used as targets for developing agents which modulate an AmgA mediated activity, e.g., binding with a protease.

The AmgA protein, fragments thereof, and derivatives and other variants of the sequence in SEQ ID NO: 2, including those set forth in SEQ ID NO: 6 and SEQ ID NO: 8, are collectively referred to as “peptides”, “polypeptides” or “proteins” of the present disclosure or “AmgA polypeptides or proteins”. “AmgA molecules” refer to AmgA nucleic acids, polypeptides, and any construct based on such molecules, such as antibodies, fusion proteins, chimerics, etc.

Allelic variants of AmgA include functional proteins. Functional allelic variants are naturally occurring amino acid sequence variants or synthetically produced sequence variants of the AmgA protein that maintain the ability to bind to at least one of the proteases of the four classes of proteases, i.e., serine, cysteine, aspartic and/or metallo proteases. Functional allelic variants may contain only conservative substitution of one or more amino acids of SEQ ID NO: 2, 6 or 8, or substitution, deletion or insertion of non-critical residues in non-critical regions of the protein.

In another aspect, the present disclosure features a method of making a fragment or analog of an AmgA polypeptide having a biological activity of a naturally occurring AmgA polypeptide. The method includes: altering the sequence, e.g., by substitution or deletion of one or more residues, of an AmgA polypeptide, e.g., altering the sequence of a non-conserved region, or a domain or residue described herein, and testing the altered polypeptide for the desired activity.

Libraries of fragments e.g., N terminal, C terminal, or internal fragments, of an AmgA protein coding sequence can be used to generate a variegated population of fragments for screening and subsequent selection of variants of an AmgA protein.

AmgA peptides of the present disclosure also include those which arise as a result of the existence of multiple genes, alternative transcription events, alternative RNA splicing events, and alternative translational and posttranslational events.

In another aspect, the present disclosure provides AmgA chimeric or fusion proteins. As used herein, an AmgA “chimeric protein” or “fusion protein” includes an AmgA polypeptide linked to a non-AmgA polypeptide. A “non-AmgA polypeptide” refers to a polypeptide having an amino acid sequence corresponding to a protein which is not substantially homologous to the AmgA protein, e.g., a protein which is different from the AmgA protein and which is derived from the same or a different organism. The AmgA polypeptide of the fusion protein can correspond to all or a portion e.g., a fragment described herein of an AmgA amino acid sequence. In one embodiment, an AmgA fusion protein includes at least one biologically active portion of an AmgA protein. The non-AmgA polypeptide can be fused to the N-terminus or C-terminus of the AmgA polypeptide.

Purified fusion proteins can be used in AmgA activity assays, (e.g., direct assays or competitive assays described in detail below), or to generate antibodies specific for AmgA proteins. The fusion protein can include a moiety which has a high affinity for a ligand. For example, the fusion protein can be a glutathione S-transferase (GST)-AmgA fusion protein in which the AmgA sequences are fused to the C-terminus of the GST sequences. Such fusion proteins can facilitate the purification of recombinant AmgA. Alternatively, the fusion protein can be an AmgA protein containing a heterologous signal sequence at its N-terminus. In certain host cells (e.g., mammalian host cells), expression and/or secretion of AmgA can be increased through use of a heterologous signal sequence.

Fusion proteins can also include all or a part of a serum protein, e.g., an IgG constant region, or human serum albumin.

In another aspect, the present disclosure provides an anti-AmgA antibody. The term “antibody” as used herein refers to an immunoglobulin molecule or immunologically active portion thereof, i.e., an antigen-binding portion. Examples of immunologically active portions of immunoglobulin molecules include F(ab) and F(ab′)₂ fragments which can be generated by treating the antibody with an enzyme such as pepsin.

The antibody can be a polyclonal, monoclonal, recombinant, e.g., a chimeric or humanized, fully human, non-human, e.g., murine, or single chain antibody. In one embodiment it has effector function and can fix complement. The antibody can be coupled to a toxin or imaging agent.

A full-length AmgA protein or antigenic peptide fragment of AmgA can be used as an immunogen to generate anti-AmgA antibodies or to identify anti-AmgA antibodies made with other immunogens, e.g., cells, membrane preparations, and the like. The antigenic peptide of AmgA should include at least about 8 amino acid residues of the amino acid sequence shown in SEQ ID NO: 2 and encompasses an epitope of AmgA. Typically, the antigenic peptide includes at least 10 amino acid residues, more typically at least 15 amino acid residues, even more typically at least 20 amino acid residues, and most typically at least 30 amino acid residues.

Antibodies reactive with, or specific for, any of these regions, or other regions or domains described herein are provided.

In one embodiment the antibody binds an epitope on any domain or region of AmgA proteins described herein. Due to the similarity of the AmgA polypeptide herein and A2M, antibodies produced in accordance with the present disclosure may also bind to A2M in humans and be utilized to treat disease states characterized by upregulation of A2M including, but not limited to, Alzheimer's disease and associated disorders.

The anti-AmgA antibody can be a single chain antibody. A single-chain antibody (scFV) may be engineered (see, for example, Colcher, D. et al., Ann. NY Acad. Sci. Jun. 30, 1999; 880:263-80; and Reiter, Y., Clin. Cancer Res. 1996 February; 2 (2):245-52). The single chain antibody can be dimerized or multimerized to generate multivalent antibodies having specificities for different epitopes of the same target AmgA protein.

An anti-AmgA antibody (e.g., monoclonal antibody) can also be used in some embodiments to isolate AmgA peptide by standard techniques, such as affinity chromatography or immunoprecipitation. Moreover, an anti-AmgA antibody can be used to detect AmgA peptide (e.g., in a cellular lysate or cell supernatant) or, as noted above, A2M, in order to evaluate the abundance and pattern of expression of the protein. Detection can be facilitated by coupling (i.e., physically linking) the antibody to a detectable substance (i.e., antibody labeling). Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

One can produce the AmgA peptide having the amino acid sequence set forth in SEQ ID NO: 2 using the isolated nucleic acid described above, for example, the nucleic acid having the nucleotide sequence of SEQ ID NO: 1, by any suitable method in any suitable expression system within the purview of one skilled in the art. Similarly, one can produce a portion of the AmgA peptide having the amino acid sequence set forth in SEQ ID NO: 6 using the isolated nucleic acid having the nucleotide sequence of SEQ ID NO: 5, or one can produce a portion of the AmgA peptide having the amino acid sequence set forth in SEQ ID NO: 8 using the isolated nucleic acid having the nucleotide sequence of SEQ ID NO: 7. Therefore, the method for producing the AmgA peptide is also within the scope of the present disclosure.

One expression system for the recombinant production of the AmgA of the present disclosure is transgenic non-human animals, wherein the desired AmgA may be recovered from the transgenic animal. In other embodiments, the nucleic acid of SEQ ID NO: 1, 5 or 7 may be subcloned into an expression vector to obtain another recombinant vector. The vector can be capable of autonomous replication or it can integrate into a host DNA. Viral vectors include, e.g., replication defective retroviruses, adenoviruses and adeno-associated viruses.

A vector can include an AmgA nucleic acid in a form suitable for expression of the nucleic acid in a host cell. Typically the recombinant expression vector includes one or more regulatory sequences operatively linked to the nucleic acid sequence to be expressed. The term “regulatory sequence” includes promoters, enhancers and other expression control elements (e.g., polyadenylation signals). Regulatory sequences include those which direct constitutive expression of a nucleotide sequence, as well as tissue-specific regulatory and/or inducible sequences. The design of the expression vector can depend on such factors as the choice of the host cell to be transformed, the level of expression of protein desired, and the like. The expression vectors of the present disclosure can be introduced into host cells to thereby produce proteins or polypeptides, including fusion proteins or polypeptides, encoded by nucleic acids as described herein (e.g., AmgA proteins, mutant forms of AmgA proteins, fusion proteins, and the like).

The recombinant expression vectors of the present disclosure can be designed for expression of AmgA proteins in prokaryotic or eukaryotic cells. In embodiments, the nucleotide sequence of the present disclosure may be cloned and incorporated into a recombinant vector comprising the nucleic acid cloned and isolated above, and optionally a regulatory sequence, such as replication region, selection marker (e.g. antibiotic resistance marker), eukaryotic cell promoter or a prokaryotic cell promoter so that the recombinant vector can be expressed in a suitable host. When used in mammalian cells, the expression vector's control functions are often provided by viral regulatory elements. For example, commonly used promoters are derived from polyoma, Adenovirus 2, cytomegalovirus and Simian Virus 40. Alternatively, the recombinant expression vector can be transcribed and translated in vitro, for example using T7 promoter regulatory sequences and T7 polymerase.

In some embodiments, a host cell includes a nucleic acid molecule described herein, e.g., an AmgA nucleic acid molecule within a recombinant expression vector or an AmgA nucleic acid molecule containing sequences which allow it to homologously recombine into a specific site of the host cell's genome. The terms “host cell” and “recombinant host cell” are used interchangeably herein. Such terms refer not only to the particular subject cell but rather also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, an AmgA protein can be expressed in bacterial cells such as E. coli, insect cells (e.g., using baculovirus expression vectors), yeast cells or mammalian cells including human cells, Chinese hamster ovary cells (CHO) or COS cells. Suitable host cells are discussed further in Goeddel, Gene Expression Technology: Methods in Enzymology 185, Academic Press, San Diego, Calif. (1990). Other suitable host cells are within the purview of those skilled in the art.

In some embodiments, nucleic acids of the present disclosure can be chosen for having codons for a particular expression system. For example, in some embodiments the nucleic acid can be one in which at least one codon, in embodiments at least about 10% of the codons, in other embodiments at least about 20% of the codons, have been altered such that the sequence is optimized for expression in E. coli, yeast, human, insect, or CHO cells.

A suitable host cell may be transformed or transfected with the recombinant vector. The transformed or transfected cells may then be cultured under conditions sufficient for expression of the AmgA protein. Finally, the expressed proteins may be recovered and purified. Methods for recovering and purifying the AmgA peptide are not limited and include, for example, various chromatographies. In some embodiments the AmgA may be expressed using histidine tag fusion protein technique, and the recovering and purifying method is performed by an affinity column.

As used herein, the terms “transformation” or “transfection” include a variety of techniques for introducing an exogenous nucleic acid into a cell (for example, eukaryotic or prokaryotic), including calcium phosphate or calcium chloride precipitation, microinjection, DEAE-dextrin-mediated transfection, lipofection, or electroporation.

Electroporation may be carried out at approximate voltage and capacitance (and corresponding time constant) to result in the entry of the DNA construct(s) into the host cells. Electroporation may be carried out over a wide range of voltages (e.g. 50 to 2,000 volts) and corresponding capacitance. Total DNA of approximately 0.1 to 500 μg is generally used.

Methods such as calcium phosphate precipitation and colubrine precipitation, liposome fusion and receptor-mediated gene delivery can also be used to transfect cells.

Thus, the present disclosure further provides methods for producing (i.e., expressing) an AmgA protein using the host cells of the present disclosure. In one embodiment, the method includes culturing the host cell of the present disclosure (into which a recombinant expression vector encoding an AmgA protein has been introduced) in a suitable medium such that an AmgA protein is produced. In another embodiment, the method further includes isolating an AmgA protein from the medium or the host cell.

In one useful embodiment, expression of proteins in prokaryotes may be carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGtX (Pharmacia Biotech Inc; Smith, D. B. and Johnson, K. S., (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

To maximize recombinant protein expression in E. coli, it may be desirable to express the protein in host bacteria with an impaired capacity to proteolytically cleave the recombinant protein (Gottesman, S., Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the present disclosure can be carried out by standard DNA synthesis techniques.

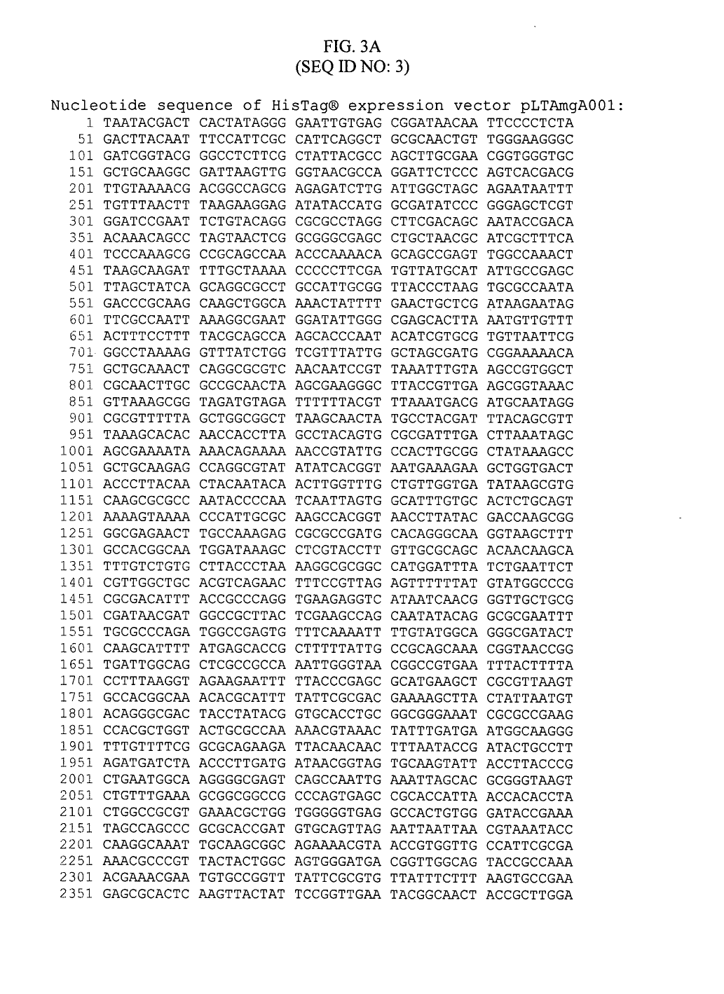

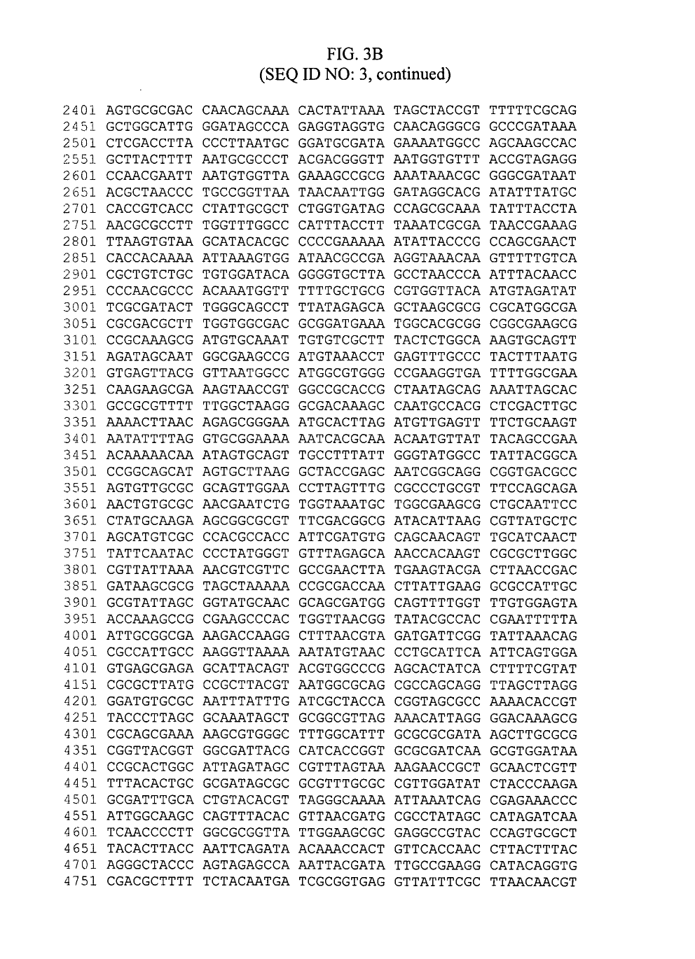

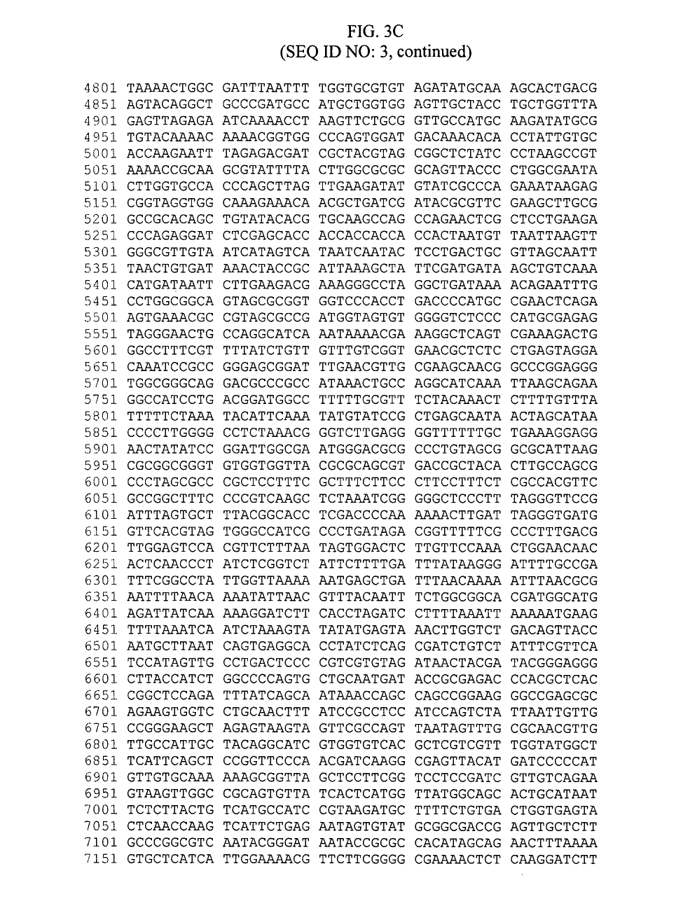

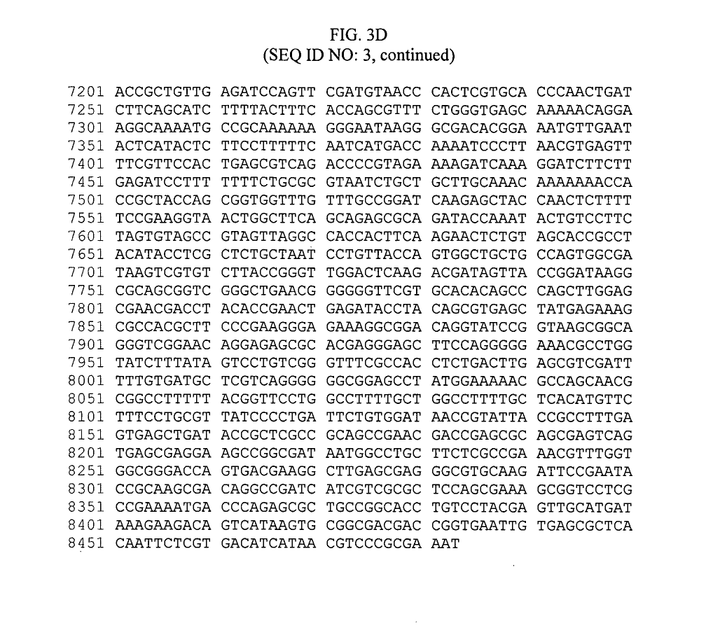

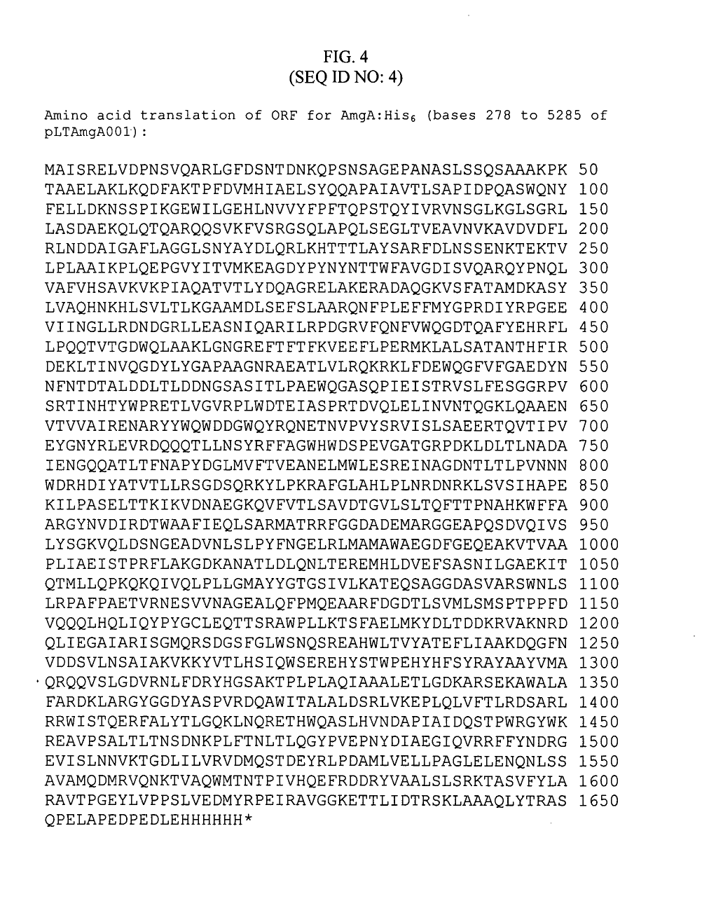

In another embodiment, the present disclosure includes a recombinant vector in which the AmgA gene is cloned into pETBlue 2 (Novagen, Madison, Wis.). Other similar vectors within the purview of one skilled in the art may be utilized to clone and express AmgA. An example of the nucleotide sequence of the present disclosure in such a vector is set forth in SEQ ID NO: 3 (FIGS. 3A-3D), wherein the nucleotide sequence of the gene for AmgA is included in a HisTag® expression vector, and is designated pLTAmgA001. The amino acid translation of the open reading frame (ORF) for the resulting protein from such a vector, designated as AmgA:His6 (bases 278 to 5285 of pLTAmgA001), is set forth in SEQ ID NO: 4 (FIG. 4).

The AmgA polypeptides of the present disclosure are likely to have a beneficial effect, for example, in the clearance of high levels of proteases which accompany certain infectious disease states, including, for example, human immunodeficiency virus, (HIV), hepatitis C virus (HCV), Rhinovirus, severe acute respiratory syndrome-associated coronavirus (SARS-CoV), certain bacterial infections including those caused by members of the genera Salmonella, Staphylococcus, Streptococcus, Mycobacterium (including Mycobacterium tuberculosis), and others, as well as other parasites. Other diseases which may be treated with protease inhibitors include, for example, certain cancers, cardiovascular diseases, neurodegenerative diseases, inflammatory/tissue injuries, or any other disease state mediated or facilitated by protease activity.

A protein prepared from this gene has great potential for commercial application. Synthetic protease inhibitors are expensive and are generally specific for a particular class of protease. Thus, mixtures of these inhibitors, sometimes referred to as cocktails, are prescribed for the treatment of various disease states, including HIV, depending upon the various proteases they are expected to encounter. A single product effective against all four protease classes could thus provide a cost-effective alternative to the expensive, and often toxic, synthetic cocktails. The A2M-like protein AmgA, RefSeq accession number ZP_{—}00315594, of Saccharophagus degradans strain 2-40 has a potential for use as such a product.

The AmgA peptides, antibodies thereto, and any other constructs based upon such peptides or nucleic acids herein, e.g., fusion proteins, chimerics, etc., may be administered as a component of a pharmaceutical composition within the purview of those skilled in the art. A pharmaceutical composition may be formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. It may be useful to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the methods of preparation may include vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods within the purview of those skilled in the art.

Toxicity and therapeutic efficacy of pharmaceutical compositions of the present disclosure can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit high therapeutic indices may be utilized. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies typically within a range of circulating concentrations that include the ED₅₀ with little or no toxicity. The dosage may vary within this range depending upon the dosage form employed and the route of administration utilized. For any compound used in the method of the present disclosure, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The protein or polypeptide can be administered one time per week for about 1 to about 10 weeks, typically about 2 to about 8 weeks, more typically about 3 to about 7 weeks. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, typically, can include a series of treatments.

The nucleic acid molecules of the present disclosure can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al., (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

Additional diagnostic and/or therapeutic techniques which can utilize the peptides of the present disclosure include, but are not limited to, removing proteases from a sample by magnetic separation using AmgA-coated magnetic beads, and “panning” with AmgA attached to a solid matrix, i.e., a plate such as a microwell plate or other similar plate, dish or tray. In addition, agarose, polyacrylamide, or sephadex beads may be utilized as well as other similar beads suitable for use in column affinity chromatography procedures, particularly for the separation of proteases from complex solutions. Such systems may be useful in diagnosing disease states characterized by increased levels of proteases, whereby proteases in a sample are removed by binding to the peptides of the present disclosure and quantified. In such a method, high levels of proteases from a sample compared to a base line level would be indicative of a disease state.

Additionally, the nucleic acids of the present disclosure and their resulting peptides may be useful in research applications. These applications, which can be easily developed into functional products, take advantage of AmgA's ability to not only inhibit a broad spectrum of proteases, but physically entrap them as well. Such applications may include, for example, removal of proteases present in fermentation cultures or that are present in the expression and purification of recombinant proteins.

For example, in some embodiments the polypeptides of the present disclosure may be utilized in fermentation procedures which use enzymes or produce proteinaceous end-products. Specific processes include enzymatic synthesis and/or degradation of chemicals, drug production, enzymatic conversion of biomass-to-energy, and various applications within the food industry. The polypeptides of the present disclosure may be utilized to remove proteases from the fermentation cultures, thereby increasing production yields by protecting the productive enzymes from proteolytic degradation. Similarly, the yields and shelf life of protein products or foodstuffs could be enhanced by removal of the attacking proteases.

In other embodiments, peptides of the present disclosure may be utilized to remove proteases formed during the expression and purification of recombinant proteins from the culture containing such recombinant proteins. Removal of these proteases from the culture reduces proteolytic degradation that would otherwise occur during purification, thus enhancing the production and recovery of the recombinant proteins.

In other embodiments, antisense molecules may be prepared which are complementary to the nucleic acids of the present disclosure. Due to the similarity between A2M and AmgA, such antisense molecules may be utilized in therapeutics to interfere with the production of A2M in those disease states characterized by high levels of A2M, including, but not limited to, Alzheimer's disease, and associated disorders.

The protein of the present disclosure, AmgA of S. degradans, has many potential advantages over commercially available A2M from human plasma. For example, because it is a prokaryotic protein, recombinant expression from E. coli should allow production of AmgA at higher purity with far lower costs than those involved with isolation from human plasma. In addition, because it is a monomeric protein, it may remain functional under conditions which would cause disassociation of the tetramers of A2M from human plasma.

The following Examples are being submitted to illustrate embodiments of the present disclosure. These Examples are intended to be illustrative only and are not intended to limit the scope of the present disclosure. Also, parts and percentages are by weight unless otherwise indicated.

### EXAMPLES

### Example 1

Saccharophagus degradans was grown in 500 ml flask cultures containing 0.2% Avicel, carboxymethylcellulose (CMC) and xylan as sole carbon and energy sources. Supernatants from the avicel, CMC, and xylan-grown cultures were concentrated to about 25 times by centrifugal ultrafiltration using Centricon™ or Microcon™ devices (Millipore). Protein concentrations were determined using the bicinchoninic acid (BCA) assay (Pierce). Briefly, the BCA assay relies on the formation of a Cu2+-protein complex under alkaline conditions, followed by reduction of the Cu2+ to Cu1+. The amount of reduction is proportional to the protein present. It has been shown that cysteine, cystine, tryptophan, tyrosine, and the peptide bond are able to reduce Cu2+ to Cu1+. BCA forms a purple-blue complex with Cu1+ in alkaline environments, thus providing a basis to monitor the reduction of alkaline Cu2+ by proteins at absorbance maximum 562 nm. The BCA assay has several advantages over other protein determination techniques, including the following: the color complex is stable; it is less susceptible to detergents, and it may be utilized over a broad range of protein concentrations.

Samples were exchanged into 100 mM Tris buffer, pH 8.5, containing 8 M urea and 10 mM dithiothreitol (DTT) and incubated for about 2 hours at 37° C. to denature and reduce the proteins. After reduction, cysteine residues were alkylated by the addition of 1M iodoacetate to a final concentration of 50 mM and incubated at 25° C. for 30 minutes. The samples were exchanged into 50 mM Tris, 1 mM CaCl2, pH 8.5 using Microcon™ centrifugal filter devices (from Millipore). The denatured, reduced, and alkylated samples were digested overnight at 37° C. using proteomics grade trypsin (Promega) at a 1:50 enzyme to substrate ratio. Digestions were stopped by the addition of 99% formic acid to a final concentration of about 1% and analyzed by RPHPLC-MS/MS using a Waters 2960 HPLC linked to a Finnagin LCQ tandem mass spectrometer. All peptide fragment masses were analyzed by the peptide analysis packages SEQUEST and MASCOT (Ducret, Van-Oostveen et al. 1998; Perkins, Pappin et al. 1999), and compared to amino acid sequence translations of all gene models in the S. degradans draft genome and to the non-redundant Mass Spectrometry Database (ftp://ftp.ncbi.nih.gov/repository/MSDB/msdb.nam). Peptide identity matches were evaluated using the accepted thresholds of statistical significance specific to each program.

The results of this experiment indicated that the AmgA protein was synthesized during growth on both forms of cellulose as well as xylan, suggesting it may be constituently expressed during growth on complex polysaccharides (CP).

### Example 2

Cloning and expression of S. degradans proteins in E. coli. AmgA was cloned as follows. Nucleotide sequences of gene models obtained from the DOE JGI's Saccharophagus degradans genome web server were used to design primers within the first and last 100 nucleotides of the AmgA ORF and 5′ restriction sites were added to the primers so as to permit in-frame cloning into pETBlue2 (Novagen Madison, Wis.). PCR reactions (50 μl) used standard parameters and conditions for tailed primers and Proof Pro® Pfu Polymerase (Continental Lab Products, San Diego, Calif.) and included S. degradans genomic DNA as the template. PCR products were ligated into pETBlue2; the nucleotide sequence of the gene for AmgA included in this HisTag® expression vector (designated pLTAmgA001) is set forth in SEQ ID NO: 3 (FIGS. 3A-3D).

### Example 3

Production and purification of recombinant proteins. Expression constructs from Example 2 were transformed into E. coli DH5α by electroporation according to the manufacturer's protocol.

Expression cultures were induced by 1 mM IPTG for four hours at 37° C. or 16 hours at 20° C. Culture pellets were harvested by centrifugation (5000×g, 20 min) and frozen overnight at −20° C. Cells were thawed on ice and suspended in 5 ml of BugBuster HT® lysis solution (Novagen; Madison, Wis.) per gram wet pellet weight. The cells were incubated for 20 minutes at room temperature and then clarified by centrifugation. The resulting supernatant was mixed with Nickel-NTA resin (QIAGEN, Valencia, Calif.) according to the manufacturer's instructions. The resin slurry was loaded onto a column and washed twice with wash buffer (50 mM NaH₂PO₄, 300 mM NaCl, 20 mM imidazole, pH 8.0). The protein was eluted at 4° C. in 50 mM NaH₂PO₄, 300 mM NaCl, 250 mM imidazole, pH 8.0. Void, wash and elution fractions were screened for production of an appropriate-sized His-tagged protein, which was confirmed by comparing pre-induced and induced (1 mM IPTG) cell lysates in western blots using 1:5000 anti-HisTag® monoclonal primary antibody (Novagen) and 1:7500 goat anti-mouse HRP conjugated secondary antibody (BioRad, Hercules, Calif.). Blots were developed colorimetrically with the OPTI-4CN kit (BioRad). Fractions containing the recombinant proteins were pooled, exchanged into Storage Buffer (20 mM Tris pH 7.4, 10 mM NaCl, 10% glycerol) using Centricon™ centrifugal ultrafiltration devices (Millipore), aliquotted and frozen at −80° C. for later use.

The amino acid translation sequence of the open reading frame (ORF) for the resulting AmgA peptide, designated AmgA:His₆ (bases 278 to 5285 of pLTAmgA001 described in Example 2 above) is SEQ ID NO: 4, which is set forth in FIG. 4.

### Example 4

A simple assay to determine the inhibitory profile of the AmgA protein could be conducted as follows. Representative proteases from each of the four classes (serine, cysteine, aspartic, and metallo proteases), would be assayed with the Protease Colorimetric Detection Kit (Sigma, catalog #PCO100) to determine their baseline activity. The assay method uses a casein substrate. As the casein is cleaved by the protease, trichloroacetic acid (TCA) soluble peptides are generated. These peptides contain tyrosine and tryptophan residues that react with the Folin & Ciocalteu's (F-C) Reagent to produce a color change. The F-C Reagent also reacts with peptides containing cystine, cysteine, and histidine residues, but to a lesser extent. The amino acids reduce the tungstate and/or molybdate in the F-C Reagent, thereby generating one or more compounds with a characteristic blue color that can be colorimetrically quantitated at 660 nm.

The proteases are then assayed with varying amounts of AmgA added. Concentrations of AmgA added can be up to the equimolar concentration of the protease being used in the inhibition assay. For example, if a 60 kDa protease is used at 5 ug/ml, AmgA will need to be added at an amount up to about 15 ug/ml, as the molecular weight of AmgA is 3 times higher. Colorometric determination of the peptides containing tyrosine and tryptophan residues that react with the Folin & Ciocalteu's (F-C) reagent to produce a color change as described above are utilized and compared with the standard curve generated above in the absence of AmgA to generate a profile of inhibitory action of AmgA.

Another set of assays will include commercially-available protease inhibitors that are specific for the protease class. For each protease the percent inhibition will be calculated and the results with AmgA will be compared to those achieved with the commercial inhibitors to evaluate the effectiveness of AmgA versus known inhibitors. Significant activity against members of multiple protease classes indicates the potential of AmgA protein for commercial development, including scale-up production and cost analyses.

The above description should not be construed as limiting, but merely as exemplifications of typically useful embodiments. It will be understood that various modifications may be made to the embodiments disclosed herein. For example, as those skilled in the art will appreciate, the specific sequences described herein can be altered slightly without necessarily adversely affecting the functionality of the nucleotide or resulting polypeptide of the present disclosure. Therefore, the above description should not be construed as limiting, but merely as exemplifications of useful embodiments. Those skilled in the art will envision other modifications within the scope and spirit of this disclosure.

What is claimed is:

1. An isolated nucleic acid molecule comprising a nucleotide sequence of SEQ ID NO: 1.

2. An isolated nucleic acid molecule of claim 1, wherein the nucleic acid molecule has at least about 50% nucleic acid sequence identity to SEQ ID NO: 1.

3. An isolated nucleic acid molecule of claim 1, wherein the nucleic acid molecule has at least about 80% nucleic acid sequence identity to SEQ ID NO: 1.

4. An expression vector comprising the nucleic acid of claim 2.

5. A host cell comprising the expression vector of claim 4.

6. The host cell of claim 5, wherein said cell is selected from the group consisting of CHO cells, insect cells, human cells, COS cells, E. coli, and yeast cells.

7. A process for producing a polypeptide comprising culturing the host cell of claim 5 under conditions suitable for expression of said polypeptide and recovering said polypeptide from the cell culture.

8. An isolated peptide having the amino acid sequence of SEQ ID NO: 2.

9. The peptide of claim 8, wherein the peptide has at least about 30% amino acid sequence identity to the amino acid sequence of SEQ ID NO: 2, and wherein the peptide possesses alpha-2-macroglobulin like activity.

10. The peptide of claim 9, wherein the peptide comprises a conservative substitution variant of the amino acid sequence having at least about 30% amino acid sequence identity to the sequence of SEQ ID NO: 2.

11. The peptide of claim 8, wherein the peptide has at least about 80% amino acid sequence identity to the amino acid sequence of SEQ ID NO: 2.

12. A pharmaceutical composition comprising the peptide of claim 10.

13. A method of treating a disease state possessing increased levels of proteases in a subject by administering to the subject the peptide of claim 10.

14. A method of removing proteases from a fermentation culture using the peptide of claim 10.

15. A method of removing proteases from a culture with a peptide of claim 10, wherein the proteases are formed during expression and purification of recombinant proteins.

16. An antibody which specifically binds to a peptide according to claim 10.

17. The antibody of claim 16, wherein said antibody is a monoclonal antibody, a humanized antibody or a single-chain antibody.

18. An isolated nucleic acid molecule comprising a nucleotide sequence selected from the group consisting of SEQ ID NO: 5 and SEQ ID NO: 7.

19. An isolated nucleic acid molecule of claim 18, wherein the nucleic acid molecule has at least about 50% nucleic acid sequence identity to the nucleotide sequence selected from the group consisting of SEQ ID NO: 5 and SEQ ID NO: 7.

20. An isolated nucleic acid molecule of claim 18, wherein the nucleic acid molecule has at least about 80% nucleic acid sequence identity to the nucleotide sequence selected from the group consisting of SEQ ID NO: 5 and SEQ ID NO: 7.

21. An expression vector comprising the nucleic acid of claim 19.

22. A host cell comprising the expression vector of claim 19.

23. The host cell of claim 22, wherein said host cell is selected from the group consisting of CHO cells, insect cells, human cells, COS cells, E. coli, and yeast cells.

24. A process for producing a polypeptide comprising culturing the host cell of claim 22 under conditions suitable for expression of said polypeptide and recovering said polypeptide from the cell culture.

25. An isolated peptide having an amino acid sequence selected from the group consisting of SEQ ID NO: 6 and SEQ ID NO: 8.

26. The peptide of claim 25, wherein the peptide has at least about 30% amino acid sequence identity to the amino acid sequence selected from the group consisting of SEQ ID NO: 6 and SEQ ID NO: 8, and wherein the peptide possesses alpha-2-macroglobulin like activity.

27. The peptide of claim 26, wherein the peptide comprises a conservative substitution variant of the amino acid sequence having at least about 30% amino acid sequence identity to the sequence selected from the group consisting of SEQ ID NO: 6 and SEQ ID NO: 8.

28. A peptide having at least about 80% amino acid sequence identity to an amino acid sequence of claim 25.

29. A pharmaceutical composition comprising the peptide of claim 27.

30. A method of treating a disease state possessing increased levels of proteases in a subject by administering to the subject the peptide of claim 27.

31. A method of removing proteases from a fermentation culture using the peptide of claim 27.

32. A method of removing proteases from a culture with a peptide of claim 27, wherein the proteases are formed during expression and purification of recombinant proteins.

33. An antibody which specifically binds to a peptide according to claim 27.

34. The antibody of claim 33, wherein said antibody is a monoclonal antibody, a humanized antibody or a single-chain antibody.
